(19) United States
(12) Patent Application Publication
(10) Pub. No.: US 2002/0169436 A1
(43) Pub. Date: Nov. 14, 2002

(54) Percutaneous method of preventing distal embolisation while maintaining adequate distal perfusion in percutaneous interventional procedures

(76) Inventors: Hitinder Gurm, Shaker Heights, OH (US); Prabhjot Singh Sandhu, Fairview Park, OH (US); Roopinderjit Malhi, Shaker Heights, OH (US)

Correspondence Address: Hitinder Gurm, 19835 Winslow Road, Shaker Heights, OH, 44122, US

(21) Appl. No.: 09/851,179

(22) Filed: May 8, 2001

(51) Int. Cl. A61M 31/00

(52) U.S. Cl. 604/509; 606/194; 604/96.01

(57) ABSTRACT

Perfusion guard is a first of a new generation of devices for interventional cardiology. It encompasses an improved debris collection system with a novel perfusion system. It uses an occlusive balloon to provide total protection from debris from interventional procedures. Distal perfusion is maintained using a variety of endogenous or exogenous perfusion substances delivered through an isolated lumen. These substances may be the patients' blood, artificial blood substitutes or biocompatible perfusion fluids. A therapeutic device or catheter may be introduced through a separate port or may be modified to be delivered over the device. Thus the device provides a safe and improved means of carrying out interventions while protecting from debris generated from these procedures and maintaining perfusion to the segments beyond the site of intervention.

### CROSS-REFERENCE TO RELATED APPLICATIONS

1: Webb J G, Carere R G, Virmani R, Baim D, Teirstein P S, Whitlow P, McQueen C, Kolodgie F D, Buller E, Dodek A, Mancini G B, Oesterle S. Retrieval and analysis of particulate debris after saphenous vein graft intervention. J Am Coll Cardiol. August 1999;34(2):468-75.

2: Stein B C, Moses J, Teirstein P S. Balloon occlusion and transluminal aspiration of saphenous vein grafts to prevent distal embolization. Catheter Cardiovasc Interv. September 2000;51(1):69-73.

3: Carlino M, De Gregorio J, Di Mario C, Anzuini A, Airoldi F, Albiero R, Briguori C, Dharmadhikari A, Sheiban I, Colombo A. Prevention of distal embolization during saphenous vein graft lesion angioplasty. Experience with a new temporary occlusion and aspiration system. Circulation. Jun. 29, 1999;99(25):3221-3.

4: Topol E J, Yadav J S. Recognition of the importance of embolization in atherosclerotic vascular disease. Circulation. Feb. 8, 2000;101(5):570-80. Review. No abstract available.

5: Gruntzig A, Hirzel H, Goebel N, Gattiker R, Turina M, Myler R, Stertzer S, Kaltenbach M. [Percutaneous transluminal dilatation of chronic coronary stenoses. First experiences]. Schweiz Med Wochenschr. Nov 4, 1978;108(44):1721-3. German.

6: Gruntzig A. [Percutaneous dilatation of experimental coronary artery stenosis- description of a new catheter system]. Klin Wochenschr. Jun. 1, 1976;54(11):543-5. German.

7: Gruntzig A, Riedhammer H H, Turina M, Rutishauser W. [A new method for the percutaneous dilation of coronary stenoses in animal experiment]. Verh Dtsch Ges Kreislaufforsch. 1976;42:282-5. German. No abstract available.

### BACKGROUND OF THE INVENTION

1. Introduction

Percutaneous interventional procedures have revolutionized the field of medicine. A common complication of these procedures is distal embolisation of plaque debris causing significant mortality and morbidity. The current technology to prevent such complications is in its infancy and has several drawbacks. We purpose the design of a new device that provides complete protection while ensuring adequate distal perfusion.

2. Background

Percutaneous coronary angioplasty was introduced as a method of opening occlusions in blocked coronary arteries. Recently its use has been extended to other vessels and bypass vein grafts. It has been increasingly recognized as a safe alternative to major surgery. Angioplasty and other interventional techniques work by disruption or displacement of the obstructing atherosclerotic plaque. A major complication of percutaneous interventional devices is distal embolisation. As the atherosclerotic plaque is displaced, it often undergoes disruption and large or small particulate matter is displaced in the circulation. This particulate matter then moves downstream and blocks off small or larger vessels. This leads to significant injury to the organ that is being perfused by that vessel. Thus a patient undergoing an intervention on the heart (native coronary artery or saphenous vein graft) suffers a myocardial infarction, one undergoing an intervention on the carotid vessels may have a stroke and a patient with a renal artery or aortic intervention may suffer from renal failure.

The last decade has seen an explosion in the number of percutaneous interventions being performed. Further there are efforts afoot to develop interventions for larger and more proximal vessels. The risk of embolisation remains a major limitation in the development of safer procedures.

Currently there are two main systems for prevention of distal embolisation; they can be categorized as balloons or occlusive devices and umbrellas. The balloons work under the principal of total occlusion of the vessel and diversion of blood flow. However the occlusion of blood flow to a critical organ even for a brief period of time may be detrimental and may itself lead to the same sequelae that it aims to prevent.

The umbrella devices consist of a mesh that can be deployed to prevent debris from floating down while maintaining perfusion. They suffer from two major flaws, they cannot stop all debris from floating past and second they easily become saturated and then retard flow and lead to the same problem that is seen with the balloon devices.

There thus remains a need to develop a safer and more perfusion friendly device, something that would be more effective at maximal capture of plaque debris and yet permitting good perfusion of the distal organ. We describe the design of a device that can provide such a function in a safe and effective manner.

### Implications

The potential uses of this device extend to almost all aspects of interventional medicine.

### Heart

The direct application of this device would be saphenous vein grafts. Approximately 33% of saphenous vein grafts become occluded by 10 years after bypass and another 33% demonstrate significant atherosclerosis. Interventions on these grafts are fraught with a large emboli burden. Further often these grafts provide life sustaining blood flow to critical cardiac segments. The current device would provide a safety net for capture of these emboli and would also ensure ongoing tissue perfusion. This would thus provide for hemodynamic stability and adequate time for maximal plaque removal.

This device could similarly be utilized for proximal coronary artery interventions by providing flow to the segment distal to the lesion being intervened on.

### Carotid Interventions

Stroke is a major source of morbidity and mortality. Stroke killed 159,791 people in 1997 and is the third largest cause of death, ranking behind “diseases of the heart” and all forms of cancer. Stroke is a leading cause of serious, long-term disability in the United States. About 4,400,000 stroke survivors are alive today. A major cause of stroke is carotid artery atherosclerosis. Recent advances in interventional medicine have allowed for interventions on atherosclerotic carotid arteries with plaque capture. Doppler studies have demonstrated ongoing plaque showers during these procedures. Approximately 1% of patients undergoing carotid interventions suffer a major stroke while a larger fraction develops some psycho-neurological impairment. This device could significantly reduce these complications.

### Stroke as a Complication of Other Interventional Procedures

Stroke is a devastating complication of interventional procedures that involve crossing the aortic arch. The usual cause is disruption of atheromatous plaque from the ascending aorta or the aortic arch or of mural thrombi within the cardiac cavity. In patients known to be at high risk for such complications, the device can be selectively used to perfuse the great vessels and thus avoid these devastating complications.

Thus this device would facilitate interventional approaches to repair of aortic aneurysms, aortic dissections and cardiac ablation procedures that involve prolonged catheterization of cardiac chambers with attendant risk of thrombosis.

### Renal Artery Interventions

Renal artery stenosis is a common cause of renovascular hypertension. It is usually resistant to medical therapy and often leads to chronic renal failure. Interventions on these lesions have been attended by risk of renal failure and often failure of improvement in the clinical condition. One possible cause for this is renal ischemia due to the procedure and due to distal embolisation. The perfusion guard is an ideal device to prevent such a complication.

### Miscellaneous

As the field of interventional medicine develops further, there will be an explosion in the nature and type of interventions being performed. If adequate perfusion could be provided to the vital organs without major risk, complex interventions could be carried out on the heart and the great vessel. Similarly, the ability to prevent embolisation and ensure perfusion would provide a safety net to intervene on vital structures currently not approached percutaneously.

### FIELD OF INVENTION

The present invention relates to the field of interventional medicine. More specifically it provides a device capable of capturing debris generated via interventional cardiovascular procedures and provides a means of perfusion distal to the procedure. The invention may be used for interventions on coronary arteries, saphenous vein grafts, carotid interventions, aorta and its branches, and interventions with high risk of arterial thromboembolism.

### BRIEF SUMMARY OF INVENTION

Perfusion guard is in brief a combination of a perfusion and a protection catheter. It uses an external source to provide perfusion using a variety of endogenous or exogenous perfusion substances. These may be the patients' blood, artificial blood substitutes or biocompatible perfusion fluids. An occlusive balloon provides the protection or the guard function with a proximal port suited to provide aspiration of the clot. While the occlusive device is deployed, perfusion is provided via the perfusion port using the above mentioned perfusion fluids. A therapeutic device or catheter may be introduced through a separate port or may be modified to be delivered over the device. Additional ports may be provided for administration of therapeutic agents or contrast dye. Thus the device serves to provide total protection from debris while providing critical perfusion to the distal segment.

This invention provides a medical device consisting of a catheter with a series of parallel hollow lumens, an occluding device and suction ports proximal to the occluding device. The central lumen is adapted to provide insertion over a guide wire and to provide a lumen for perfusion fluid delivery. The other lumen provides a channel for insertion of various catheters and devices and can also serve as a suction port for removal of debris. Additional channels may be added to obtain pressure measurements, insert additional diagnostic or interventional devices and administer medications or diagnostic contrast media.

### BRIEF DESCRIPTION OF THE SEVERAL VIEWS OF THE DRAWING

FIG. 1 Perfusion guard deployed beyond occlusive plaque and perfusion provided via central lumen prior to therapeutic intervention.

FIG. 2 Embodiment of device with pressure transducers proximal and distal to the occluding member.

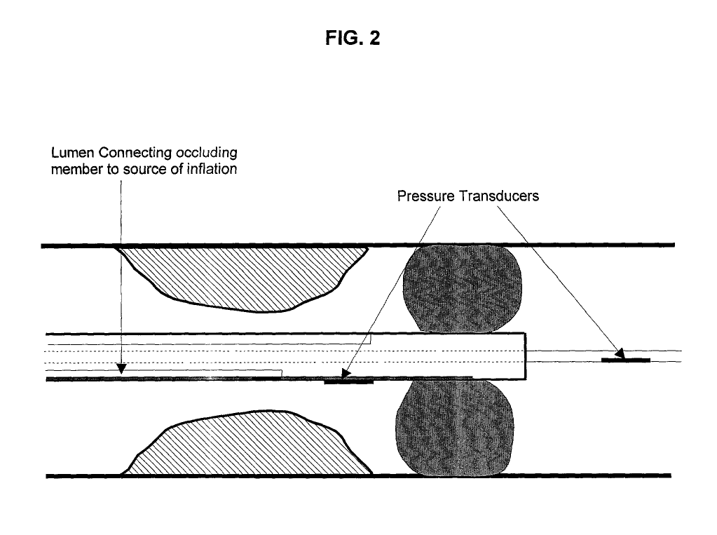

FIG. 3 View of device showing port for introduction of therapeutic device or for administration of medication or angiographic device.

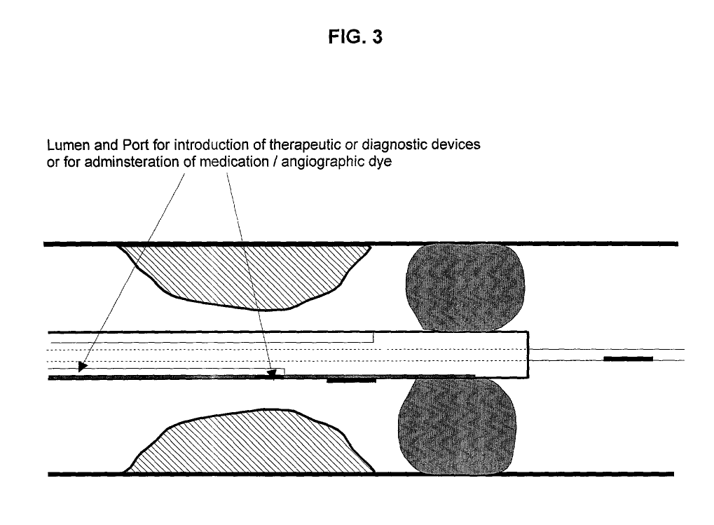

FIG. 4 Angioplasty being performed while protection and perfusion is being provided by the device.

FIG. 5 Stent being deployed while protection and perfusion is being provided by the device.

### DETAILED DESCRIPTION OF THE INVENTION

Perfusion guard is in brief a combination of a perfusion and a protection catheter. It uses an external source to provide perfusion using a variety of endogenous or exogenous perfusion substances. These may be the patients' blood, artificial blood substitutes or biocompatible perfusion fluids. A single occlusive balloon provides the protection or the guard function with a proximal port suited to provide aspiration of the clot. The same or a different port may be used to introduce other devices or catheters thus providing the means to combine it with almost all kinds of current devices. Thus it serves to protect from debris while providing critical perfusion to the distal segment.

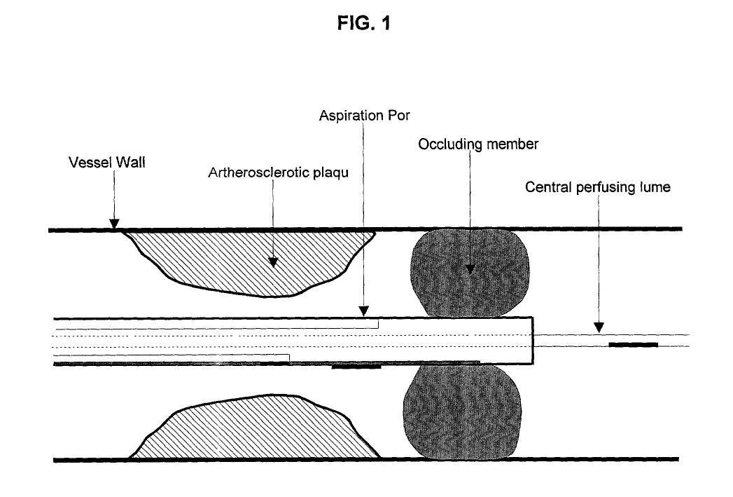

One embodiment of the device consists of a medical device with a single occluding balloon. The central lumen of the catheter communicates with the end distal to the balloon at one end and the proximal end at the other. (FIG. 1) This lumen is adapted for insertion over a guide wire and also provides a means to provide perfusion. The occluding balloon communicates with an inflation lumen and an inflation port at the proximal end of the catheter. Another lumen communicates proximally and provides a suction and flushing lumen ending proximal to the occluding member. The same lumen may be used for introduction of a therapeutic catheter.

In another embodiment, the catheter has two lumens ending proximal to the balloon, one providing a port for therapeutic catheter deployment and the other for drug or contrast administration or debris aspiration.

In another embodiment, the catheter has three lumens, one for device deployment, one for continuous aspiration and the third for drug or angiographic contrast administration.

In another embodiment, pressure transducers are deployed at both the distal tip and proximal to the occluding balloon to measure the perfusion pressure in both segments of the vessel, proximal and distal to the occluding balloon.

In another embodiment there are two occluding members, the second occluding member is proximal to the aspiration port. This thus helps to isolate a segment of the blood vessel, preventing blood from flowing in and at the same time allowing for complete removal of debris.

The invention provides means of safely perfusing various organs while protecting distal embolisation of debris. In the first method, the catheter is introduced beyond an occlusion in the vessel and distal perfusion started under low pressure. The occluding balloon is slowly inflated while the distal perfusion is increased to an acceptable level. Acceptable occlusion of the occluding catheter is confirmed using angiographic dye. Subsequently a therapeutic device is inserted either through the therapeutic port or independently and the planned intervention carried out. The debris generated is removed using the distal suction port. The therapeutic catheter is removed at the end of the procedure and the port used to provide flushing to ensure complete debris removal. Once adequate clearance has been obtained, the occluding balloon is deflated and catheter removed after adequate endogenous perfusion is maintained.

In another method, the same principal is applied to the coronary arteries, in another to a saphenous vein graft and in another to carotid artery and in other to any branch of the aorta.

In another method, three catheters are inserted into the great arteries arising from the aortic arch and perfusion provided to the brain while debris-generating procedures are carried out on the proximal aorta or the heart. The occlusion balloon is carefully deployed in each while perfusion maintained to the distal segment using the previously described biocompatible perfusion fluid pumped under controlled settings via the central lumen. The procedure is carried out using therapeutic catheters inserted independently or via the therapeutic port and at the end of the procedure, the debris collected is aspirated and the occluding balloons deflated while adequate perfusion is confirmed using the distal manometers or independent devices.

This method may be further modified to provide protection to other vital organs like the kidneys by deploying additional catheters. In a modification of these methods an umbrella may be deployed over the catheter to provide two methods of debris collection. The reminder of the procedure would be carried out as previously described. The umbrella would be deployed prior to the therapeutic catheter insertion and removed prior to the balloon deflation.

In another modification, the therapeutic device is advanced over the catheter and allows for plaque removal while continuous aspiration is provided via the suction port

It will be understood that the device would provide the means to carry out complex interventional procedures in humans with a safe collection of the embolic debris and provide adequate tissue perfusion to prevent distal organ dysfunction. The device would minimize the attendant ischemia and its associated hemodynamic complications associated with the use of the current devices. It could be used with any interventional procedure where there is an expectation of distal embolisation and hence could be used in any angiographic suite in any hospital.

The human arterial tree consists of the ascending aorta the aortic arch and the descending aorta and the various branches that arise from it. Blood usually moves away from the aortic arch and flows along a pressure gradient. The ascending aorta gives rise to two coronary arteries which supply the heart and the aortic arch gives rise to the three great vessels, the right brachiocephalic, the left common carotid and the left subclavian artery. These three branches provide blood supply to upper half of the body and the brain. The descending aorta gives rise to multiple branches that perfuse various body organs before dividing into the common iliacs which after further division give rise to the femoral arteries which supply the legs. Since the arterial tree is devoid of valves, a catheter inserted in any of the major branches of aorta can be advanced into any of the other branches.

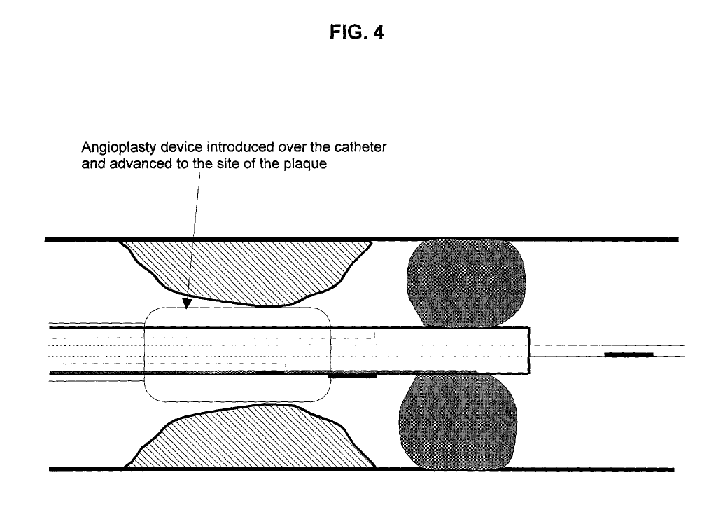

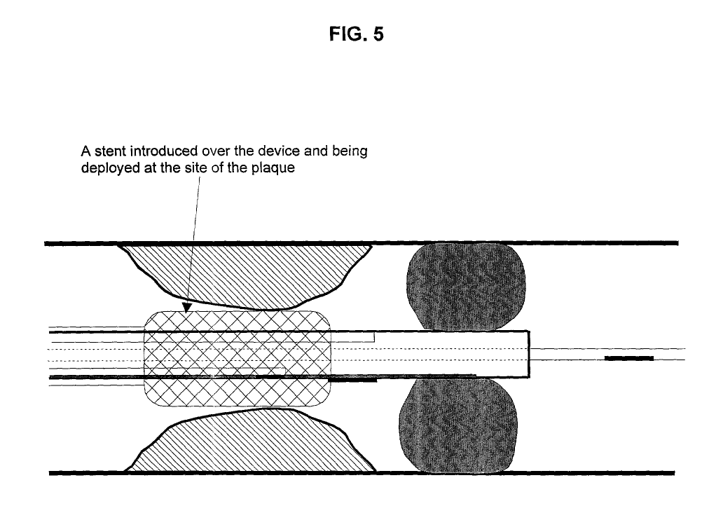

Access to the arterial tree is obtained via the arm or the leg for the purpose of various angiographic procedures and has been previously well described. Further a variety of methods of intervention have been described that are intended to open up obstruction in the arterial tree. The major drawback of these interventions is the risk of embolisation, i.e. the debris released by the process of intervention floats down stream and causes interruption of blood flow to critical segments of vital organs. Current systems of debris collection fail to capture all debris and this is predominantly due to failure to dam the blood flow totally for a prolonged period of time. The invention describes a new method for deploying a total barrier to the debris and yet being able to provide a flow of vital perfusion fluid through the device to the organs upstream. In the first embodiment of the device (FIG. 1), the device is introduced over a guide wire through an arterial access sheath or an arteriotomy and advanced beyond an occlusion in the target artery. The guide wire is removed and the perfusion port connected to an external pump that would be able to supply a steady supply of the perfusion liquid. The occluding member would than be deployed, thereby isolating the vessel beyond the target occlusion. The therapeutic device is then deployed either independently or via an additional port (FIG. 4 & FIG. 5) and the plaque intervened on using the various forms of therapeutic devices. The therapeutic catheter is then withdrawn and the debris aspirated using an external suction source.

Since the nature of the procedure is virtually the same wherever arterial blood flows, the vessel may be any branch of the aorta or of its branches or sub branches or a graft placed to bypass any of these. The perfusion fluid would be a blood substitute or a synthetic solution capable of providing the necessary nutrients to the target organ and would be perfused using an external pump. In another embodiment of the invention, the patient's blood obtained via cannulation from another site would be directed into the perfusion channel using an external connection.

In another embodiment of the device, angiographic dye would be injected using the aspiration port or a separate port to ensure adequate isolation of the arterial segments proximal and distal to the occluding member

In another embodiment, the device would be equipped with manometers both distal to the transducer and proximal to the transducer. This would ensure that adequate pressure is maintained by the perfusing system.

In another embodiment, the therapeutic catheter is advanced through one port, a cleaning liquid is perfused through another while the cleaning liquid and the debris is continuously removed via another port.

In another embodiment, the device would have a therapeutic device deployed over it. Thus once adequate perfusion is obtained and the occluding member deployed, the therapeutic device would be advanced to the correct position and the intervention carried out. The device would have an infusion port for a continuous infusion of a cleaning liquid and another port for continuous aspiration of the cleaning liquid and the debris.

In another embodiment, the device would have two occluding members, one that is just proximal to the perfusion channel and the second that is proximal to the aspiration channel. The deployment of the two occluding members would isolate the segment of the vessel between the two occluding members. Then the therapeutic device is deployed, and all the debris generated is collected between the two occluding members. Once it has been aspirated and the segment washed out with a cleaning liquid, the occluding members are removed followed by the removal of the catheter.

In another embodiment, the device would be deployed further away from the site of the intervention. It would for example be used to isolate the renal arteries when there is the risk of debris embolisation to the kidneys or the great vessels arising from the arch to isolate and protect the brain. Thus the device would be advanced into the vessel perfusing the critical organ, the perfusion fluid infused using the perfusion system and the occluding member deployed to prevent debris from floating by, while a procedure is carried out further upstream.

The device would thus combine protection and perfusion functions and hence make safer many of the current cardiovascular interventions. However, its use may also lead to execution of many interventions hitherto believed to be too risky. Thus its usage is likely to exceed the indications described above, creating newer indications and modifying the field of interventional medicine in the process.

The length of the catheter will generally be between 10 and 200 cm preferably between 30 and 150 cm. The inner diameter of the perfusing lumen would be of the order of 0.01-2.0 cm and the diameter of the expanded occluder would be between 0.2-5.0 cm. The foregoing ranges are set solely for the purpose of illustrating typical device dimensions. The actual dimensions of the device constructed according to the principles of the present invention may obviously vary outside the listed ranges without departing from these basic principles.

Although the foregoing invention has, for the purpose of clarity and understanding been described in some detail by way of illustration and example, it will be obvious that certain changes and modifications may be practiced which still fall within the scope of the appended claim.

What is claimed is

1. a method for capturing emboli distal to an interventional procedure and maintaining adequate tissue perfusion using an external pump, the method comprising the steps of
providing a catheter having a proximal end, a distal end and a series of lumens in between, the distal end having an occluding member and a series of lumens opening via single or multiple ports proximal to the occluding member:
Inserting the device beyond an occlusion in the vessel in an ante grade direction
Connecting the central lumen to an external pump via the distal end and providing supply of a perfusing liquid to the artery
Expanding the occluding member to occlude the blood flow proximal to the occluding member
Using a therapeutic instrument on the vessel by inserting it through one of the proximal lumens or independent of the catheter
Aspirating the debris from the procedure using via a lumen proximal to the occluding member

2. The method of claim 1
Where the occluding member is a balloon

3. The method of claim 1
Where there is another balloon proximal to the aspiration port

4. The method of claim 1
Where an umbrella device can be deployed over the catheter to provide another method of debris capture in addition to or in lieu of the occluding member.

5. The method of claim 1
Where the perfusion fluid is patient's own blood obtained via cannulation of an artery away from the site of intervention.

6. The method of claim 1
Where the perfusion fluid is a substitute for blood.

7. The method of claim 1
Where the therapeutic instrument is an angioplasty device

8. The method of claim 1
Where the therapeutic device is an atherectomy device

9. The method of claim 1
Where the therapeutic device is an ultrasonic device

10. The method of claim 1
Where the therapeutic device is a stent delivery device

11. The method of claim 1
Where the therapeutic device can be deployed over the device

12. The method of claim 1
Where angiographic dye can be injected to visualize the vessel proximal or distal to the catheter

13. The method of claim 1
Where medications may be administered via the perfusion fluid

14. The method of claim 1
Where medications may be administered proximal to the occluding member.

15. The method of claim 1
Where the device is used to provide protected perfusion while the intervention is carried out further upstream.
